(19) United States
(12) Patent Application Publication
(10) Pub. No.: US 2022/0401258 A1
(43) Pub. Date: Dec. 22, 2022

(54) A FEMALE CONDOM

(71) Applicant: Om Prakash GARG, Nashik (IN)

(72) Inventor: Om Prakash GARG, Nashik (IN)

(21) Appl. No.: 17/777,445

(22) PCT Filed: Aug. 21, 2020
(86) PCT No.: PCT/IB2020/057853

§ 371 Date: May 17, 2022

(30) Foreign Application Priority Data
Nov. 23, 2019 (IN) 201921047949

(51) Int. Cl. A61F 6/06

(52) U.S. Cl. CPC A61F 6/065

(57) ABSTRACT

The present disclosure relates to the field of condoms (100) and envisages a female condom (100) which comprises a sheath (102) configured in an oblong shape, an internal retainer (104) and an external retainer (106). The sheath (102) is configured to expand into an elongated configuration when a penis is inserted therein, and prevent contact of body fluids during coitus. The internal retainer (104) is located at the closed end of the sheath (102), and is configured to be squeezed before being inserted into the vagina. The internal retainer (104) is configured to fit over the cervix for minimizing the chances of removal of the condom (100) from the vagina. The external retainer (106), having an octagonal configuration, is configured on the open end of the sheath (102) to facilitate resting of said condom (100) on the vulva for avoiding slippage of the condom (100) during coitus.

### FIELD

The present disclosure relates to the field of condoms.

### BACKGROUND

The background information herein below relates to the present disclosure but is not necessarily prior art.

Among the innumerable forms and methods of contraception, the condom is widely commercialized. During sexual intercourse between the male and the female, the condom acts as a physical barrier for preventing unwanted pregnancies and venereal diseases. The condom also avoids the complications associated with intrauterine devices and birth control pills.

Typically condoms are designed to be worn by men and are hence, referred to as male condoms. However, the male condoms have a major drawback, i.e., they interfere with the normal progression of coitus. Use of such condoms requires the male user to be aroused first, followed by an interruption to open the condom package and apply the condom onto the penis of the user. As a result, a significant amount of time is consumed due to which the interest of the partner decreases. Moreover, condoms are usually packaged in a lubricating fluid, and are hence cold to touch and discouraging to its use. Further, the male condoms sometimes cause discomfort to the wearer if they are too tight or too loose.

There is, therefore, felt a need for a female condom that alleviates the drawbacks mentioned hereinabove.

### OBJECTS

Some of the objects of the present disclosure, which at least one embodiment herein satisfies, are as follows:

An object of the present disclosure is to ameliorate one or more problems of the prior art or to at least provide a useful alternative.

An object of the present disclosure is to provide a female condom.

Another object of the present disclosure is to provide a female condom that makes coitus relatively more pleasurable.

Yet another object of the present disclosure is to provide a female condom that is user friendly.

Other objects and advantages of the present disclosure will be more apparent from the following description, which is not intended to limit the scope of the present disclosure.

### SUMMARY

The present disclosure relates to a female condom. The condom comprises a sheath, an internal retainer and an external retainer. The sheath is configured in an oblong shape, and has a closed end and an open end. The sheath is configured to remain in a contracted shape when inserted into a vagina, and is further configured to expand into an elongated configuration when a penis is inserted therein. The sheath is configured to retain the ejaculated seminal fluid therein. The sheath is further configured to act as a barrier between the male and female mucosa to prevent contact of body fluids during coitus. The internal retainer is located at the closed end of the sheath. The internal retainer is configured to be squeezed before being inserted in the vagina. The internal retainer is further configured to fit over the cervix and act as an anchor to minimize the chances of removal of the condom from the vagina. The external retainer is configured on the open end of the sheath to facilitate resting of the condom on the vulva. The external retainer has an octagonal configuration for avoiding slippage of the condom during coitus.

### BRIEF DESCRIPTION OF THE ACCOMPANYING DRAWING

A female condom of the present disclosure will now be described with the help of the accompanying drawing, in which:

FIG. 1 illustrates an isometric view of the female condom;

FIG. 2 illustrates a front view of the female condom of FIG. 1;

FIG. 3 illustrates a side view of the female condom of FIG. 1;

FIG. 4 illustrates a top view of the female condom of FIG. 1; and

FIG. 5 illustrates a bottom view of the female condom of FIG. 1.

### LIST OF REFERENCE NUMERALS

100—Female condom
102—Sheath
104—Inner retainer
106—Outer retainer

### DETAILED DESCRIPTION

Embodiments, of the present disclosure, will now be described with reference to the accompanying drawing.

Embodiments are provided so as to thoroughly and fully convey the scope of the present disclosure to the person skilled in the art. Numerous details, are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, elements, modules, units and/or components, but do not forbid the presence or addition of one or more other features elements, components, and/or groups thereof.

When an element is referred to as being “mounted on,” “engaged to,” “connected to,” or “coupled to” another element, it may be directly on, engaged, connected or coupled to the other element. As used herein, the term “and/or” includes any and all combinations of one or more of the associated listed elements.

Terms such as “inner,” “outer,” “beneath,” “below,” “lower,” “above,” “upper,” and the like, may be used in the present disclosure to describe relationships between different elements as depicted from the figures.

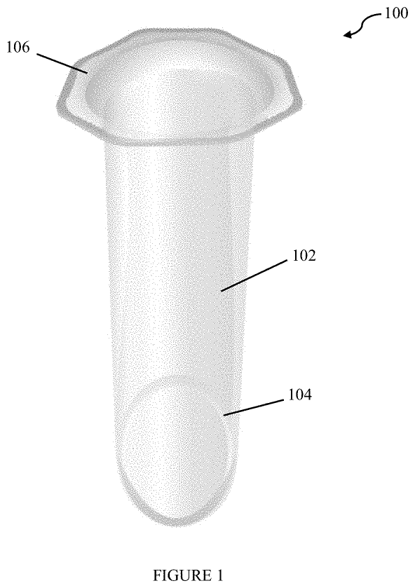

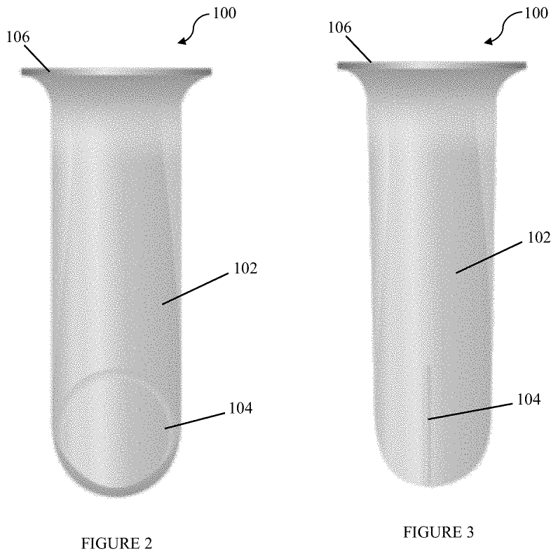

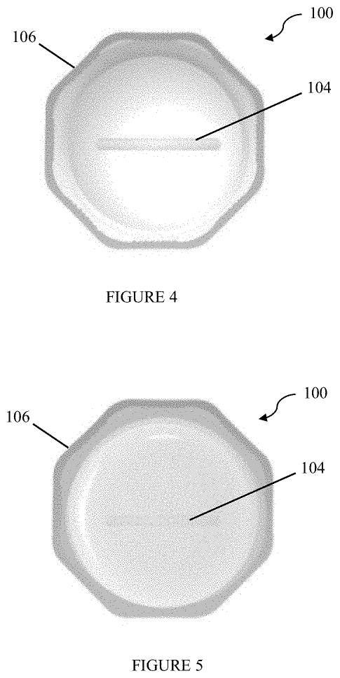

A female condom (100) of the present disclosure will now be described with reference to FIG. 1 through FIG. 5. The female condom (100) (hereinafter referred to as ‘condom (100)’) is configured to be inserted into the vagina, and retain the seminal fluid therein, thus protecting the user from unintended pregnancies. The condom (100) further is configured to act as a physical barrier between the male and female genitals to prevent transfer of sexually transmitted infections (STIs), including HIV.

The female condom (100) comprises a sheath (102), an internal retainer (104) and an external retainer (106). The sheath (102) is configured in an oblong shape, and has a closed end and an open end. The sheath (102) is configured to remain in a contracted shape when inserted into a vagina. The sheath (102) is further configured to expand into an elongated configuration when a penis is inserted therein. The sheath (102) is configured to retain the ejaculated seminal fluid therein, thereby eliminating the chances of being impregnated. The sheath (102) is further configured to act as a barrier between the male and female mucosa to prevent contact of body fluids during coitus. The internal retainer (104) is located at the closed end of the sheath (102). The internal retainer (104) is configured to be squeezed before being inserted in the vagina. The internal retainer (104) is further configured to fit over the cervix and act as an anchor to minimize the chances of removal of the condom (100) from the vagina. The external retainer (106) is configured on the open end of the sheath (102) to facilitate resting of the condom (100) on the vulva. The external retainer (106) is a ring having an octagonal configuration for avoiding slippage of the condom (100) during coitus.

In an operative configuration, the internal retainer (104) is squeezed and inserted in the vagina, making sure that the internal retainer (104) does not twist. The internal retainer (104) is inserted in the vagina till it rests behind the pubic bone and fits on the cervix, thus enabling the internal retainer (104) to act as an anchor and minimizing the chances of removal of the condom (100). On the other hand, the external retainer (106) rests on the vulva is such a way that the external retainer (106) covers the vulva more specifically, parts of the perineum and labia, thereby making sure that the female condom (100) does not slip away and is kept in position in spite of the heavy forces exerted during the coitus. When the penis is inserted in the vagina, the sheath (102) expands to accommodate the penis therein. After coitus, the external retainer (106) needs to be twisted before pulling the condom (100) out, in order to prevent the seminal fluid from leaking out. The condom (100) has to be discarded after a single use.

In an embodiment, the sheath (102) is manufactured from natural rubber latex, more specifically, by dipping a mold into the latex solution several times to form a multilayered sheath. In one embodiment, the sheath (102) is a bi-layered sheath. Thereafter the sheath (102) is compounded to provide strength for bearing the brunt of the expansive and abrasive forces that are exerted during coitus. In another embodiment, the sheath (102) is of polymer such as nitrile or polyurethane.

The sheath (102) is pre-lubricated by a silicon-based lubricant. In an embodiment, the silicon-based lubricant is poly dimethyl siloxane also known as silicon oil.

The external retainer (106) is configured on the open end of the sheath (102) in such a way that the ring does not come in contact with the human body when in use. This configuration is achieved by integrating the external retainer (106) with the sheath.

In an embodiment, the internal retainer (104) and the external retainer (106) are of polymer. In an embodiment, the external retainer (106) is made of polyethylene. In another embodiment, the internal retainer (104) is a ring manufactured using polyurethane.

The female condom (100) can be used with any type of lubricant like water-based, silicone-based or oil-based lubricants. The female condom (100) can be put in any time before coitus, usually at least eight hours before coitus. As a result, the contact between the genital members can be comfortable at body temperature and without the need for interruptions for wearing the condom (100), thus making coitus relatively more pleasurable.

Further, the female condom (100) covers a larger surface area of the female genital, thereby decreasing the chances of skin-to-skin transmission of venereal diseases to the minimal.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

### TECHNICAL ADVANCEMENTS

The present disclosure described herein above has several technical advantages including, but not limited to, the realization of a female condom that:
makes coitus more pleasurable; and
Is user friendly.

The embodiments herein and the various features and advantageous details thereof are explained with reference to the non-limiting embodiments in the following description. Descriptions of well-known components and processing techniques are omitted so as to not unnecessarily obscure the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A female condom (100) comprising:
a sheath (102) configured in an oblong shape, and having a closed end and an open end, said sheath (102) configured to remain in a contracted shape when inserted into a vagina, and further configured to expand into an elongated configuration when a penis is inserted therein, said sheath (102) configured to retain the ejaculated seminal fluid therein, and further configured to act as a barrier between the male and female mucosa to prevent contact of body fluids during coitus;
an internal retainer (104) located at the closed end of said sheath (102), said internal retainer (104) configured to be squeezed before being inserted in the vagina, said internal retainer (104) further configured to fit over the cervix and act as an anchor to minimize the chances of removal of said condom (100) from the vagina; and
an external retainer (106) configured on the open end of said sheath (102) to facilitate resting of said condom (100) on the vulva, said external retainer (106) having an octagonal configuration for avoiding slippage of the condom (100) during coitus.

2. The female condom (100) as claimed in claim 1, wherein said sheath (102) is a multilayered sheath manufactured from natural rubber latex which is compounded to provide strength for bearing the expansive and abrasive forces exerted during coitus.

3. The female condom (100) as claimed in claim 2, wherein said sheath (102) is a bi-layered sheath.

4. The female condom (100) as claimed in claim 1, wherein said sheath (102) is pre-lubricated by a silicon-based lubricant.

5. The female condom (100) as claimed in claim 4, wherein said silica-based lubricant is poly dimethyl siloxane.

6. The female condom (100) as claimed in claim 1, wherein said internal retainer (104) is a ring manufactured using polyurethane.

7. The female condom (100) as claimed in claim 1, wherein said external retainer (106) is integrated with said sheath (102).

8. The female condom (100) as claimed in claim 1, wherein said external retainer (106) is made of polyethylene.
